(19) United States
(12) Patent Application Publication
(10) Pub. No.: US 2024/0033276 A1
(43) Pub. Date: Feb. 1, 2024

(54) COMPOSITION FOR PRETERM INFANTS TO REDUCE TIME TO FULL ENTERAL FEEDING

(71) Applicant: SOCIETE DES PRODUITS NESTLE S.A., Vevey (CH)

(72) Inventors: JELENA BUNCIC-MARKOVIC, St-Prex (CH); YIPU CHEN, La Tour-de-Peilz (CH)

(21) Appl. No.: 18/255,751

(22) PCT Filed: Dec. 2, 2021
(86) PCT No.: PCT/EP2021/083926

§ 371 Date: Jun. 2, 2023

(30) Foreign Application Priority Data
Dec. 4, 2020 (EP) 20211788.3

(51) Int. Cl. A61K 31/702; A61P 1/00; A23L 33/00; A23L 33/125

(52) U.S. Cl. CPC A61K 31/702; A61P 1/00; A23L 33/40; A23L 33/125

(57) ABSTRACT

The present invention concerns a stable aqueous composition comprising at least one human milk oligosaccharide for use in reducing time to reach full enteral feeding in preterm infants; wherein the composition is administered as soon as possible after birth between day 1 and 7 of life. Aqueous composition for use according to present invention is a milk fortifier or a supplement.

### FIELD OF THE INVENTION

The present invention concerns a stable aqueous composition comprising human milk oligosaccharides to reduce time to full enteral feeding for infants. The invention also relates to the use of such aqueous composition as a milk fortifier or supplement for feeding low birthweight infants (LBW infants), very low birthweight infants (VLBW infants), extremely low birthweight infants (ELBW infants) and preterm infants for improving oral tolerance.

### BACKGROUND OF THE INVENTION

Mother's milk is recommended for all infants. However, in some cases breast feeding is inadequate or unsuccessful for medical reasons or the mother chooses not to breast feed. Infant formulae have been developed for these situations. Fortifiers and supplements have also been developed to enrich mother's milk or infant formula with specific ingredients.

The importance of oligosaccharides which are contained in human breast milk (human milk oligosaccharides, HMOs) is well recognized in the scientific community as being key to support digestive health, gut and/or mucosal maturation, and/or immune maturation in infants. Accordingly, where the feeding of an infant is deprived of such nutrients (as the infant receives infant formulas not containing HMOs) or where the amount of HMOs in the human breast milk or formula the infant receives are not adequate to his needs, a composition providing HMOs in the form of a supplement to be administered to the infant or dissolved into his feeding would be desirable.

In particular, an aqueous liquid composition comprising HMOs would be needed.

EP 2 768 311 relates to a synthetic nutritional composition for use in the promotion of intestinal angiogenesis and of nutrient absorption and of enteral feeding tolerance and/or in the prevention and/or treatment of intestinal inflammation, such as necrotizing enterocolitis, and/or in the recovery after intestinal injury and/or surgery. This composition is for use in mammals, preferably in humans, more preferably in infants.

WO 2010/063601 relates to probiotic composition for use in achieving full enteral feeding in infants having a low birth weight.

Liquid aqueous compositions for enteral nutrition need to be microbiologically safe for preterm infants.

Accordingly, it is an object of the present invention relates to a method for feeding low birthweight preterm infants (LBW infants), very low birthweight preterm infants (VLBW infants), extremely low birthweight preterm infants (ELBW infants) for improving oral tolerance. In the present study feeding tolerance has been clinically demonstrated for the first time by achieving decreased time to reach full enteral feeding.

### SUMMARY OF THE INVENTION

Accordingly, in one aspect the present invention provides an aqueous composition comprising at least one human milk oligosaccharide for use in reducing time to reach full enteral feeding in preterm infants; wherein the composition is administered as soon as possible after birth between day 1 and 7 of life.

This result is surprising given that early supplementation of human milk oligosaccharides was effective among population of predominantly human milk fed preterm infants.

In another aspect, the present invention provides an aqueous composition as a milk fortifier or supplement for preterm infants who were low birthweight infants (LBW infants), very low birthweight infants (VLBW infants), extremely low birthweight infants (ELBW infants).

### DETAILED DESCRIPTION OF THE INVENTION

### Definitions

A “preterm” or “premature” means an infant or young child who was not born at term. Generally it refers to an infant or young child born prior 37 weeks of gestation.

An “infant having a low birth weight” means a preterm having a body weight below 2500 g (5.5 pounds) either because of preterm birth or restricted fetal growth. It therefore encompasses:
infant or young child who has/had a body weight from 1500 to 2500 g at birth (usually called “low birth weight” or LBW)
infant or young child who has/had a body weight from 1000 to 1500 g at birth (called “very low birth weight” or VLBW)
infant or young child who has/had a body weight under 1000 g at birth (called “extremely low birth weight” or ELBW).

Within the context of the present invention, the term “monosaccharide” indicates carbohydrates containing from 3 to 6 carbon atoms. They can be polyhydroxy aldehydes or polyhydroxyketones depending on whether they comprise either an aldehyde or a ketone group, along with —OH substituted carbons in a chain. Polyhydroxy aldehydes are called “aldoses”. Polyhydroxyketones are called “Ketoses”. Non limiting examples of 6 carbon monosaccharide (hexose) are: allose, altrose, glucose, mannose, gulose, idose, galactose, talose, psicose, fructose, sorbose and tagatose. Non limiting examples of 5 carbon monosaccharide (pentose) are: ribose, arabinose, xylose, lyxose, ribulose and xylulose.

Within the context of the present invention, the term “oligosaccharide” indicates a linear or branched saccharide polymer containing a small number (typically two to ten) of simple sugars (5 or 6 membered monosaccharides as above defined). Non-limiting examples of such oligosaccharides are: “fucosylated oligosaccharide” that are based on lactose, meaning an oligosaccharide having at least one fucose residue and a glucose at the reducing end. Some examples are 2′-FL (2′ fucosyllactose), difucosyllactose (DFL, also known as LDFT, Lactodifucosyltetraose), LNT (lacto-N-tetraose), para-lacto-N-neohexaose (para-LNnH), LNnT (lacto-N-neotetraose)and any combination thereof.

Within the context of the present invention, the expressions “fucosylated oligosaccharides comprising a 2′-fucosyl-epitope” and “2-fucosylated oligosaccharides” encompass fucosylated oligosaccharides with a certain homology of form since they contain a 2′-fucosyl-epitope, therefore a certain homology of function can be expected.

Within the context of the present invention, the expression “at least one fucosylated oligosaccharide” and “at least one N-acetylated oligosaccharide” means “at least one type of fucosylated oligosaccharide” and “at least one type of N-acetylated oligosaccharide”. In one embodiment the oligosaccharide of the present invention is a combination of 2-FL and LNnT in a ratio ranging from 1:20 to 2:1, preferably 1:15 to 1:1, most preferably of 1:10 to 1:2. In a particularly advantageous embodiment, this ratio is 2:1 or around 2:1. In one embodiment the ratio of 2-FL:LNnT is 10:1.

Within the context of the present invention, the term “aqueous compositions” identifies liquid compositions which may be solutions and/or dispersions of the at least one oligosaccharide in an aqueous mean. In one embodiment the aqueous composition of the present invention can be obtained by a process as described in WO 2020/120426.

The term Full Enteral Feeding (FEF) is defined as end of parenteral nutrition and when minimum enteral intake of 150 ml/kg/day is attained. The term pre Full Enteral Feeding (pre-FEF) refers to the period from the day of birth till FEF is attained. Parenteral nutrition (consisting of fats, amino acids and sugars) is administered through intravenous route while enteral feeding (consisting for human milk or preterm formula) is administered through digestive tract. Study intervention was administered through the enteral route after enrolment (before FEF has been reached) until neonatal unit discharge.

Within the context of the present invention, the term “pH modulator” indicates a substance which is capable of affecting (i.e. decreasing, increasing or stabilizing) the pH of an aqueous solution. Non-limiting examples of pH modulators are strong and mild acids (organic or inorganic), acidic oligosaccharides, strong and mild bases (organic or inorganic) as well as buffers (organic or inorganic). Non limiting examples of organic acids are: Citric acid, Phosphoric acid, Lactic acid and sialic acid. Non limiting examples of inorganic bases are: potassium hydroxide (KOH) and sodium hydroxyde (NaOH). Non limiting examples of acidic oligosaccharides are sialic acid [N-acetyl-neuraminic acid (Neu5Ac)] or uronic acids (glucuronic acid, galacturonic acid).

in one embodiment the pH modulator is a “buffer” and/or “buffering agent”. Non-limiting examples of buffering agents are: citric acid, acetic acid, phosphate salts (sodium or potassium). Non limiting examples of buffering solutions are: Phosphate buffer (based on 2 phosphates salts, for example sodium phosphate monobasic and sodium phosphate dibasic) and citrate-phosphate buffer (for example McIlvaine buffer—based on citric acid and disodiumphosphate)

Within the context of the present invention, the term “fortifier” refers to a composition which comprises one or more nutrients having a nutritional benefit for infants.

By the term “milk fortifier”, it is meant any composition used to fortify or supplement either human breast milk, infant formula, growing-up milk or human breast milk fortified with other nutrients. Accordingly, the human milk fortifier of the present invention can be administered after dissolution in human breast milk, infant formula, growing-up milk or human breast milk fortified with other nutrients or otherwise it can be administered as a stand alone composition.

When administered as a stand-alone composition, the human milk fortifier of the present invention can be also identified as being a “supplement”. In one embodiment, the milk fortifier of the present invention is a supplement.

By the term “human milk fortifier”,_it is meant any composition used to fortify or supplement human breast milk, or human breast milk fortified with other nutrients.

Within the context of the present invention, the expression “composition having a pH ranging from value X to value Y” identifies compositions having a pH range which has one specified value within the indicated range (extremes X and Y of the range being included) as well as compositions having a pH which varies within the indicated range (extremes X and Y of the range being included).

### EMBODIMENTS OF THE INVENTION

### Oligosaccharides

In one embodiment, the aqueous composition according to the present invention comprises two or more human milk oligosaccharides for use in reducing the time to full enteral feeding (FEF) in preterm infants. The oligosaccharide(s) comprises 2′-FL, DFL, LNnT, LNT and combinations thereof.

In one embodiment, the present invention provides an aqueous composition as above defined which comprises 2′-FL and LNnT in a ratio of 10:1 for use in reducing the time to FEF in preterm infants.

In another embodiment, the present invention provides an aqueous composition at pH ranging from 4 to 7 as above defined which comprises 2′-FL and

LNnT in a ratio of 10:1 wherein the composition is administered as soon as possible after birth between day 1 and 7 of life. In one embodiment the dosage amounts for pre-FEF ranges from 0.21-0.63 g/d and dosage amounts for FEF ranges from 0.43-0.82 g/d.

In one embodiment the reduction to FEF is at least two days.

In one embodiment, the present invention provides an aqueous composition as above defined which comprises 2′-FL, DFL and LNT in a ratio of 10:1:3.33.

In one embodiment, the aqueous composition according to the present invention comprises four human milk oligosaccharides is 2′-FL, DFL, LNnT and LNT.

In one embodiment of the present invention, the aqueous composition does not have sialylated oligosaccharides or probiotics.

pH range

In one embodiment, the aqueous composition according to the invention has a pH ranging from 4 to 7.

In a further embodiment, the aqueous composition according to the invention has a pH ranging from 5.8 to 6.3 or 5.9 to 6.2 or around 6.

Aqueous compositions

In one embodiment, the present invention provides an aqueous composition comprising at least one oligosaccharide having a glucose unit at the reducing end, wherein the pH of such aqueous composition ranges from 4 to 7, for example from to 6.3 or from 5.9 to 6.2, and the composition doesn't comprise other nutrients in addition to the at least one oligosaccharide.

In one embodiment, the present invention provides an aqueous composition comprising at least one human milk oligosaccharides, a pH modulator and wherein the pH of such aqueous composition ranges from 5.5 to 6.5, for example from 5.8 to 6.3 or from 5.9 to 6.2, and the composition doesn't comprise other nutrients in addition to the oligosaccharide.

In one embodiment, the present invention provides an aqueous composition comprising at least one human milk oligosaccharides, a pH modulator, a buffering agent and wherein the pH of such aqueous composition ranges from 5.5 to 6.5, for example from 5.8 to 6.3 or from 5.9 to 6.2, and the composition doesn't comprise other nutrients in addition to the oligosaccharide.

In another embodiment, the present invention provides an aqueous composition comprising two human milk oligosaccharides, wherein the pH of such aqueous composition ranges from 5.5 to 6.5, for example from 5.8 to 6.3 or from 5.9 to 6.2, and the composition doesn't comprise other nutrients in addition to the oligosaccharides.

In another embodiment, the present invention provides an aqueous composition comprising two human milk oligosaccharides, a pH modulator and wherein the pH of such aqueous composition ranges from 5.5 to 6.5, for example from 5.8 to 6.3 or from 5.9 to 6.2, and the composition doesn't comprise other nutrients in addition to the oligosaccharides.

In another embodiment, the present invention provides an aqueous composition comprising two human milk oligosaccharides, a pH modulator, a buffering agent and wherein the pH of such aqueous composition ranges from 5.5 to 6.5, for example from 5.8 to 6.3 or from 5.9 to 6.2, and the composition doesn't comprise other nutrients in addition to the oligosaccharides.

In another embodiment, the present invention provides an aqueous composition comprising two human milk oligosaccharides, a pH modulator, a buffering agent and wherein the pH of such aqueous composition ranges from 5.5 to 6.5, for example from 5.8 to 6.3 or from 5.9 to 6.2, the oligosaccharides are 2′-FL and LNnT and the composition doesn't comprise other nutrients in addition to the oligosaccharides.

In one embodiment, the present invention provides an aqueous composition comprising at least one human milk oligosaccharide at a concentration ranging from 5 to 50% w/w of the composition.

In another embodiment, the present invention provides an aqueous composition comprising at least one human milk oligosaccharide at a concentration ranging from 8 to 35% w/w of the composition.

In a still further embodiment, the present invention provides an aqueous composition comprising at least one human milk oligosaccharide having a glucose unit at the reducing end at a concentration ranging from 10 to 30% w/w of the composition.

In one embodiment, the present invention provides an aqueous composition comprising at least one human milk oligosaccharides at a concentration ranging from 8 to 35% w/w of the composition, a pH modulator and wherein the pH of such aqueous composition ranges from 5.5 to 6.5, for example from 5.8 to 6.3 or from 5.9 to 6.2.

In one embodiment, the present invention provides an aqueous composition comprising at least one human milk oligosaccharides at a concentration ranging from 8 to 35% w/w of the composition, a pH modulator, a buffering agent and wherein the pH of such aqueous composition ranges from 5.5 to 6.5.

In another embodiment, the present invention provides an aqueous composition comprising two human milk oligosaccharides at a concentration ranging from 8 to 35%w/w of the composition, wherein the pH of such aqueous composition ranges from 5.5 to 6.5, for example from 5.8 to 6.3 or from 5.9 to 6.2.

In another embodiment, the present invention provides an aqueous composition comprising two human milk oligosaccharides at a concentration ranging from 8 to 35%w/w of the composition, a pH modulator, a buffering agent and wherein the pH of such aqueous composition ranges from 5.5 to 6.5, for example from 5.8 to 6.3 or from 5.9 to 6.2, the oligosaccharides are 2′-FL and LNnT.

In a further embodiment, the present invention provides an aqueous composition comprising five human milk oligosaccharides at a concentration ranging from 8 to 35%w/w of the composition, a pH modulator, a buffering agent and wherein the pH of such aqueous composition ranges from 5.5 to 6.5, for example from 5.8 to 6.3 or from 5.9 to 6.2, the oligosaccharides are 2′-FL, 3′-SL, 6′-SL, DFL and LNT.

In a still further embodiment, the present invention provides an aqueous composition comprising six human milk oligosaccharides at a concentration ranging from 8 to 35%w/w of the composition, wherein the pH of such aqueous composition ranges from 4.5 to 6.5, for example from 5.8 to 6.3 or from 5.9 to 6.2.

In one embodiment, the present invention provides an aqueous composition comprising at least one oligosaccharide having a glucose unit at the reducing end at a concentration ranging from 8 to 35%w/w of the composition, wherein the pH of such aqueous composition ranges from 5.5 to 6.5, for example from 5.8 to 6.3 or from 5.9 to 6.2, and the composition doesn't comprise other nutrients in addition to the at least one oligosaccharide.

In another embodiment, the present invention provides an aqueous composition comprising two human milk oligosaccharides at a concentration ranging from 8 to 35%w/w of the composition, wherein the pH of such aqueous composition ranges from 5.5 to 6.5, for example from 5.8 to 6.3 or from 5.9 to 6.2, and the composition doesn't comprise other nutrients in addition to the oligosaccharides.

### Format

By having a liquid form, aqueous compositions according to the present invention present some particular benefits. For example, they may be more conveniently packed to deliver calibrated drops of a certain weight or volume.

In some embodiment, aqueous compositions of the present invention may be packed in single doses in such a way that calibrated drops of a certain weight or volume are delivered while avoiding contamination of the remaining liquid due to manipulation and subsequent uses.

In one embodiment, the liquid aqueous composition according to the present invention is presented in single dose units which are packed in plastic material. In one embodiment, such plastic material is flexible and squeezable. In one embodiment, such plastic material may be polypropylene (PP) or Polyethylene (PE). In one embodiment, polypropylene may be low density (LD PE) or high density (HD PE),In addition, aqueous compositions are easy to mix with compositions to be fortified, whereas the powder ones can, in some cases, form lumps.

### Supplements

In one embodiment, the aqueous composition according to the present invention and above described is a supplement. In such embodiment, the aqueous composition of the invention is administered as a standalone composition. In such embodiment, the aqueous composition of the invention is administered as a standalone composition and is packed in single doses.

### Fortifiers

In one embodiment, the aqueous composition according to the present invention is a milk fortifier. In such embodiment, the aqueous composition of the invention may be packed in single doses.

### Experimental Section

Objectives and Study: Low birthweight (LBW) preterm infants are susceptible to developmental programming of adverse health outcomes and abnormal ex-utero growth patterns. Poor early growth within the neonatal unit is associated with later developmental delays. Early introduction and faster progression to full enteral feeding (FEF) is preferred, since prolonged parenteral feeding predisposes the preterm child to extrauterine growth restriction, sepsis, liver problems and exacerbated gut immaturity. This randomized, double-blind, placebo-controlled trial of LBW preterm infants evaluated effects of HMO supplementation on feeding tolerance, growth and safety from 7 centers in France.

Methods: Preterm infants between 27 and 33 weeks of gestational age with birth weight <1700g, who are younger than 7 days of age were randomized as early as possible after birth to receive HMO supplement comprising of 2′FL and LNnT (n=43) in 10:1 ratio (0.34 and 0.034 g/kg body weight/day, respectively) or an isocaloric Placebo supplement (n=43) consisting of only glucose (0.14 g/kg/day) from enrolment until discharge from the neonatal unit. Infants received the interventional product as soon as 24 hours of trophic feeding was possible and within 7 days of birth (Pre-FEF Day 1), until Discharge from the Neonatal unit. The pre-FEF period (which was considered to be the duration between Pre-FEF Day 1 and FEF day 1) was variable to allow for the gradual increase in enteral feeding until full enteral feeding (defined as end of parenteral nutrition and minimum enteral intake of 150 ml/kg/day) is attained. Primary outcome was feeding tolerance as demonstrated by non-inferiority (NI) of difference in time to reach FEF from birth (NI margin=+4 days) between HMO and Placebo groups. Study period from enrolment until day when FEF had been achieved is considered Pre-FEF period.

| | | | Post-discharge |
|---|---|---|---|
| Pre-FEF period | | FEF period | observational follow-up period |
| Randomization | EXPL: | liquid supplement containing 2 HMOs at dose | Observation only; |
| | | of 0.374 g/kg/day | no product given |
| | CTRL: | liquid placebo containing glucose only | |

→

| | | | | | | | | |
|---|---|---|---|---|---|---|---|---|
| Birth | Pre-FEF | FEF Day1 | FEF Day7 | FEF Day14 | FEF Day21 | Discharge from | 2-months | 12-months |
| | Day1 | | | | | Neonatal Unit | CA | CA |

Results: Mean chronological age at enrolment in HMO and Placebo groups were 6.3 and 6.2 days, respectively. Non-inferiority in time to reach FEF was achieved in HMO vs. Placebo (treatment difference −2.16, 95% CI −5.33, 1.00 days, p<0.001). Similar results were observed in the PP population. HMO group achieved FEF two days earlier from birth vs. Placebo (LS Means: 12.15 vs. 14.32 days) although difference did not reach statistical significance (p=0.177).

Mean daily gastric residual volumes (ml/kg/day) were not statistically significantly different between the HMO and Placebo groups in any time period in the ITT population (pre-FEF period, FEF Days 2−7, 8−14, 15-21, or Day 21-Discharge) but the direction of effect was towards lower gastric residual volume in the HMO group (overall treatment difference from baseline to discharge −0.37 (95% CI −1.28; 0.55), p=0.403). Mean stool frequency (stools/day) was generally similar between the HMO and Placebo groups within each of the same time periods with no statistically significant differences. The treatment difference from baseline to discharge was in the direction of higher mean stool frequency in the HMO group compared to the Placebo (overall treatment difference 0.24 (95% CI −0.34; 0.82, p=0.411). Stool consistency as measured on a validated five-point scale (1=watery, 2=runny, 3=mushy soft, 4=formed, 5=hard) and then averaged to obtain mean stool consistency per day. As expected, mean stool consistency moved in the direction of firmer stools in both groups over time with mean consistency of 3.09 (SD 0.70) and 3.11 (SD 0.92) in the HMO and Placebo groups, respectively, from baseline to FEF Day 1 and 3.69 (SD1.15) and 3.87 (SD 1.59) from FEF Day 21 to Discharge. The overall mean consistency from Baseline to Discharge was similar between the groups and trended in the direction of softer stools in the HMO group (overall treatment difference 0.24 (95% CI −0.34; 0.82), p=0.411). Stool consistency as measured on a validated five-point scale (1=watery, 2=runny, 3=mushy soft, 4=formed, 5=hard) and then averaged to obtain mean stool consistency per day. Mean stool consistency was similar in both groups over time with mean consistency of 2.97 (SD 0.37) and 2.98 (SD 0.45) in the HMO and Placebo groups, respectively, from baseline to FEF Day 1 and 3.07 (SD 0.23) and 3.04 (SD 0.35) from FEF Day 21 to Discharge. The overall mean consistency from Baseline to Discharge was similar between the groups and trended in the direction of firmer stools in the HMO group (overall treatment difference 0.13 (95% CI −0.04; 0.30), p=0.139). Overall, GI tolerance measures were comparable in the HMO and Placebo groups indicating that HMO supplementation is well-tolerated in preterm infants.

Thus HMO supplementation is safe and well-tolerated in preterm infants. When given as soon as possible after birth, HMO supplementation reduced time to full enteral feeds.

1. A method for reducing the time to reach full enteral feeding in preterm infants comprising administering an aqueous composition comprising at least one human milk oligosaccharide to an infant at a time period of between day 1 and 7 of life.

2. Method according to claim 1 wherein the composition is a milk fortifier or a supplement.

3. Method according to claim 1 wherein the composition pH ranges from 4 to 7.

4. Method according to claim 1 wherein decreasing the time to full enteral feeding can be commenced in infants that are born before the end of the 37 th week of pregnancy and infants that have a weight of less than 2500 grams at birth and infants that have a weight of less than 1500 grams at birth and/or infants that have a weight of less than 1000 grams at birth at birth.

5. Method according to claim 1 wherein the composition further comprises a pH modulator.

6. Method according to claim 1 wherein the at least one oligosaccharide is selected from the group consisting of: 2′-FL, DFL, LNnT and LNT.

7. Method according to claim 1 which comprises only two oligosaccharides as human milk oligosaccharides comprising 2′-FL and LNnT at ratio of 10:1.

8. Method according to claim 1 wherein the composition does not comprise other nutrients in addition to the at least one oligosaccharide.

9. Method according to claim 1 which comprises oligosaccharides at a concentration ranging from 8 to 35% w/w of the composition.

10. Method according to claim 1 wherein the composition is used as a milk fortifier or supplement for preterm infants who were low birthweight infants (LBW infants).
